(19) United States
(12) Patent Application Publication
(10) Pub. No.: US 2005/0113740 A1
(43) Pub. Date: May 26, 2005

(54) Device for administering or ingesting fluid or powder-type substances in addition to a replacement element for said device

(76) Inventor: Siegfried Oberhofer, Ingolstadt (DE)

Correspondence Address: DORITY & MANNING, P.A., POST OFFICE BOX 1449, GREENVILLE, SC, 29602-1449, US

(21) Appl. No.: 10/506,704

(22) PCT Filed: Nov. 30, 2002
(86) PCT No.: PCT/EP02/13534

(30) Foreign Application Priority Data
Mar. 8, 2002 (DE) 10210122.1

(51) Int. Cl. A61M 1/00

(52) U.S. Cl. 604/27

(57) ABSTRACT

The invention relates to a device for administering or ingesting fluids or powder-type substances (S) for a person, especially medical substances and/or food for children. Said device comprises a receiving unit (2) for receiving the substance(s) which are to be administered and a discharge unit (4) which is connected to the receiving unit (2) and which has a discharge opening (8) for discharging the substance (S) to the individual. Said device (1) comprises, at least in parts, an outer form which is not functionally related to the administration. The invention also relates to replacement elements for said device, in addition to the use of said device for administering medicaments and/or food for children.

The invention is relative to a device for administering or for ingesting fluid or powdery substances to a person, especially medical substances and/or food to children.

The administration or receiving of fluid substances can take place in many ways. For example, for the oral administration of liquid medicines customary household spoons or measuring cups are used. These devices are of a very functional nature. In the case of bitter-tasting substances such as, e.g., cod-liver oil or medicines it is frequently difficult for the parent or the medical personnel to get the child to be supplied to take the medicine. Even medicines or injections in gaseous or aerosol form have the same difficulties. An administration method that does justice to the child and is pedagogically valuable is lacking in this regard. Even the feeding of small children with, e.g., pap or other foodstuffs is often very difficult, which can have causes of various natures.

The present invention has the problem of improving with simple means the administration of fluids or powdery substances to a person, especially to children.

This problem is solved in that the device of the initially cited type comprises a receiving unit for receiving the substance to be administered and a dispensing unit connected to the receiving unit which dispensing unit comprises a dispensing opening for dispensing the substance to the person, which device has at least in parts an outer form not directly functionally related to the administration.

The receiving unit can receive the substance either directly or indirectly. This means for the latter instance that the receiving unit can also receive an insert or a container for receiving the substance or also receive an insert for receiving a container with a substance contained in it.

A device is made available by means of the combination of the invention of the at least in parts not directly functionally designed, three-dimensional design of the device with a receiving unit and a dispensing unit connected to the latter, which device is ideally suited for administering in particular pharmaceutical products in liquid, gaseous and/or powdery or aerosol form. When designing the device with a shape that is attractive in particular to children, an emotional bond of the child to the device can be achieved in that the child does not perceive the device to be particularly purposeful, that is, that it is not directly associated with the administration of the substance that might not taste very good to him, which lowers the inhibition threshold and the resistance to ingesting this substance or this food.

As an alternative to an oral administration of the substance the device can also be used for injections, e.g., insulin or vaccines. In this instance it is advantageous if an injection device is present in the vicinity of the dispensing opening for injecting the substance with a needle or by overpressure.

It is especially advantageous to design the device at least in parts to have a shape attractive for children. An example of this is a design in the form of an animal. Small children in particular feel strongly drawn to certain animal figures, e.g., elephants, bears, giraffes and monkeys. It is therefore purposeful to select a shape stemming from the animal world. In the alternative, the figure of a comic figure or of a fantasy creature can be used, e.g., a known figure such as Mickey Mouse, Donald Duck, teletubbies or the like. Shapes in the form of pieces of fruit, model cars, etc. are also possible.

A simple administration of the questionable substance is facilitated if the shape has design elements that make it easier to grasp and hold the device. In an especially preferred embodiment the extremities and/or other body parts of the animal, comic figure or fantasy creature can serve for this. E.g., when using an animal it can be grasped by two paws or on its neck area by the grownup or even by the child himself.

It is especially preferable if the dispensing opening is designed to be introduced into the mouth of the person concerned. Especially in the case of children the direct contact with the dispensing opening can help overcome inhibition thresholds. If the lips of the child can surround the dispensing opening, on the one hand an advantageous suction reflex can be utilized and on the other hand a clean, drop-free administration is possible. For example, the dispensing opening can be designed as the trunk of an elephant or as an animal snout. However, even when designing the device of the invention for injection the dispensing opening can be formed from a body part of an animal, comic figure or fantasy creature in order to reduce the fear of the child regarding the injection.

It is especially preferred if openings for dispensing the substance on the one hand and for receiving and introducing the substance, an insert and/or a container into the receiving unit are not identical. This design facilitates the manipulation since an insert and/or a container can be introduced into the receiving unit, e.g., via a relatively wide receiving opening in a simple manner whereas the administration can take place via a narrow dispensing opening. A simpler and more precise dosing of the substance to be administered can also be achieved by an adult in this instance, as will be explained in detail further below. On the other hand the variant with identical receiving and dispensing openings is on the whole simpler to manufacture.

It is preferable if the receiving unit or the dispensing unit and especially preferable if both units are substantially not visible from the outside in order that the functional aspect recedes far behind the visual and emotional aspect and the device tends to be viewed as a toy rather than a dispensing device.

In an advantageous embodiment of the invention the receiving unit and the dispensing unit are designed in one piece and thus are particularly easy to manufacture. Also, this prevents deposits from forming on connection points which would otherwise be present between the two units.

Alternatively, the two units are designed as independent parts that can preferably be non-positively connected to each other, especially with a clamp-, plug-, screw- or bayonet connection. Thus, given the appropriate design of the remaining device parts, the two units can be separated from one another, if necessary, and replaced either individually or at the same time, e.g., in the case of damage, excessive contamination, etc. without having to discard the entire device.

A first insert can also be inserted into the receiving unit, which insert can receive the substance to be administered or a container containing the substance. Also, a second insert can be inserted into the dispensing unit through which insert the substance can pass to the dispensing opening. In these embodiments the receiving unit and the dispensing unit are preferably permanently integrated into the device whereas the first and the second inserts are replaceable and thus facilitate the managing of the device. For example, different inserts can be used for different substances to be administered. If the insert or inserts need to be cleaned, it is not absolutely necessary to clean the entire device. Even if the first and/or the second insert is/are damaged, only they need to be replaced.

Preferably the first and the second insert can be connected non-positively to one another, especially by a clamp-, plug-, screw- or bayonet connection.

Alternatively, the first and the second inserts are designed in one piece so that they can be inserted and removed jointly into the receiving unit and the dispensing unit.

It is especially preferable if the receiving unit and the dispensing unit are connected positively and non-positively by a plastic surrounding them substantially completely (except, e.g., for openings for filling in and dispensing the substance, for inserting containers or inserts, etc.), which plastic was applied around the two units with an injection-molding method, blow-out method or rotation method. The negative for the tool corresponds with advantage to the three-dimensional shape of the device of the invention.

In an alternate variant of the invention the receiving unit, dispensing unit and the part of the device comprising the outer shape are designed altogether in one piece. In this extreme and simplest case the device of the invention can consist of only one element if possible insert and/or closures, that will be discussed further below, are disregarded. In this instance the receiving unit and the dispensing unit are formed from the inner walls of the device.

In another alternative the device is substantially designed as a hollow body into which the receiving unit and the dispensing unit are, e.g., suspended or clipped in. The hollow body can be designed, e.g., to be substantially rigid or also inflatable.

It is advantageous and preferable if the surfaces of the device coming in contact with the substance to be administered are designed to be food-resistant. Accordingly, the receiving unit, if it is provided for being directly filled with the substance, as well as the dispensing unit and/or one or several inserts for the receiving unit and/or the dispensing unit are provided at least on the appropriate contact surfaces with a food-resistant material. A suitable coating can be used for this purpose. Alternatively, solid materials can be used, e.g., glass, porcelain and/or a suitable plastic. Aluminum or high-grade steel can also be used.

However the entire device can also be manufactured from one or several of the previously cited materials.

Alternatively, the device elements not coming in contact with the substance consist of materials that are not absolutely food-resistant, e.g., appropriate plastic, cellulose, ceramic material, wood or the like; however, metals, including fine-grade steel and aluminum, can also be used. It is especially advantageous if the device part with the outer shape is substantially manufactured from these materials in as far as it does not make appreciable contact with the substance to be administered.

It is especially advantageous if the receiving unit can be designed with a first closure unit in a reversibly closable manner. The first closure unit can close in particular a receiving opening or a filling opening of the receiving unit so that in this manner the substance or an insert or container containing the substance, that is provided for being introduced into the receiving unit or into an appropriately designed insert, can not flow or fall out of the device.

It is also possible to design the dispensing opening with a second reversibly closable closure unit. This can effectively prevent fluid still present in the device from running out. Upon another administration the second closure unit can be removed again.

The first and/or the second closure unit are preferably designed as a cover, plug, movable closure unit or as a self-closing unit. The cover can be threaded, for example. The first or the second closure unit can alternatively be opened and/or closed by actuating a button or switch or the like. The closure unit can have a round or square form or some other suitable form. It can also be designed as a pacifier or the like.

If the cited receiving opening of the receiving unit is identical with the dispensing opening of the dispensing device, the first and the second closure units can also be identical.

It can also be provided that the first and/or the second closure unit(s) is/are integrated into the second and/or the first closure unit(s). For example, the first closure unit can be designed as a screwable cover in which, for example, a, e.g., shiftable or rotatable second closure unit is integrated for freeing and closing the dispensing opening. Such a design is known, e.g., from commercial drinking bottles for children.

The device in accordance with the invention is especially preferably designed for administering liquid as well as gaseous substances. To this end the receiving unit can be closed with different closure units, in which case one closure unit can advantageously be used for dispensing liquid substances and another closure unit can advantageously be used for dispensing gaseous substances from a gas-tight container inserted in the receiving unit. Moreover, when administering a liquid, it can be placed, e.g., directly into the receiving unit or also into a replaceable insert or container, whereas the gaseous substance to be administered and located in a gas container can be introduced into an appropriately adapted insert or directly into the receiving unit.

Furthermore, it is advantageous if the first and/or the second closure unit or units and an insert or a container can be connected to each other, e.g., plugged in to each other, so that this combination can be introduced as an entire unit into the receiving unit or the dispensing unit. Alternatively, the first or the second dispensing unit and an insert or a container are designed in one piece.

The device of the invention preferably comprises a transport device that transports the substance to be administered to the dispensing opening upon active actuation by the user. In addition, it is necessary, especially when administering medicine in most instances, to precisely dose the amount to be dispensed. Accordingly, there is the possibility of making an appropriate dosing with the transport device.

In an embodiment of the invention that is advantageous in this connection the transport can be realized with the aid of a pump mechanism. Such a mechanism is known, e.g., from inhaler sprays used with asthma patients.

When administering, e.g., pulpy foods the transport device can be designed as a manually operable piston that can shift in particular in the receiving unit.

An alternative pump mechanism can be realized if the walls of the receiving unit are designed to be flexible so that the substance to be administered can escape from the receiving unit via the dispensing opening upon being appropriately loaded with pressure, preferably exerted with the fingers.

In an advantageous embodiment the transport device is integrated at least partially in the first closure unit. For example, an actuating element extends past the first closure element and can be pressed down in order to administer the substance. Upon such an actuation a part of a container inserted into the receiving unit or the entire container is moved downward so that an opening that is preferably on the container bottom is freed through which, e.g., an aerosol can exit from the container and be orally applied via the dispensing opening. Various generally known constructions can be used for such a pump mechanism. Note in this connection, e.g., the known aerosol containers for the above-mentioned ingestion of, e.g., asthma agents.

The plurality of shapes for the three-dimensional outer form of the device can be expanded if the dispensing unit comprises a hollow line connecting the receiving unit and the dispensing unit to one another. For example, the hollow line can then be provided in the elephant trunk if the device of the invention has the shape of an elephant. This also includes embodiments in which the hollow line establishes a connection between an insert and/or container that can be inserted into the receiving unit and between the dispensing opening.

In order to prevent residual liquid from running out of the device the hollow line is designed so that it runs upward at least in sections toward the dispensing opening when the device is placed on a horizontal surface. There is a possibility in this instance that at least the conduit section located directly in front of the dispensing opening has this upward course. It can be sufficient if this section has only a slight rise of a few degrees relative to the horizontal. When a liquid substance is used, residual amounts of the substance cannot run forward or drop out.

Alternatively or additionally, at least one flow regulating means arranged in the device is provided for preventing an undesired exiting of the substance from the dispensing opening. Such a flow regulating means can be opened in a preferred embodiment of the invention by pressure and/or suction so that the substance can flow in the direction of the dispensing opening. If there is no pressure or suction the flow regulating means blocks the dispensing of the substance. A suction can be applied, e.g., by introducing the dispensing opening into the mouth and by a subsequent sucking. A loading with pressure can be realized, e.g., by the above-described pump mechanism.

In a related special embodiment of the invention a flow regulating means is designed as a thin membrane (like a soft palate) that can be deflected by pressure and/or suction and can be arranged with preference in the hollow line. The membrane can remain untouched, e.g., during the manufacture of the hollow line by milling from the one side and of the receiving unit from the other side as a separating element between these two.

Alternatively, flow regulating means that can be actuated especially by hand can be realized, e.g., slides that can block, e.g., the hollow line. Likewise, a flow regulating means can be coupled to the above-cited transport devices. When the transport device is actuated, e.g., a valve can be opened.

It can also be advantageous to provide sealing means that prevent the fluid to be administered from passing to undesired locations in the receiving unit. Such locations can be present in particular in the receiving unit if an insert or container is used so that the substance does not come in direct contact with the walls of the receiving unit. It is then the goal to assure that the substance passes via the shortest path to the dispensing opening and is not distributed in the receiving unit or seeps into cracks [scratches]. It is therefore advantageous to use, e.g., O-rings that rest on the one hand on the inner wall of the receiving unit and on the other hand on the outer wall of an insert or of a container.

A simple means of handling the device can be realized, e.g., if the receiving unit or an insert or container inserted in it can be filled from above with the substance when the device is placed on a horizontal surface.

It is particularly advantageous if the entire device is designed to be washing-machine-resistant and reusable. This assures on the one hand a simple handling and cleaning of the device. One the other hand a permanent emotional bond of the child to the particular figure or device can develop.

The above-cited replaceable elements, that can be designed as receiving unit, dispensing unit and/or as inserts for these units, represent separate aspects of the invention. Even the closure units can be designed as replaceable parts or additional parts or accessories. The cited parts can be removed if damaged or unattractive and be replaced by new inserts without having to dispose of the entire device.

Likewise, the use of a device such as described above for the administration of medical substances and/or foods to children is a component of the present invention.

Other advantageous embodiments of the invention are characterized by the features of the subclaims.

The invention is explained in detail in the following using the figures.

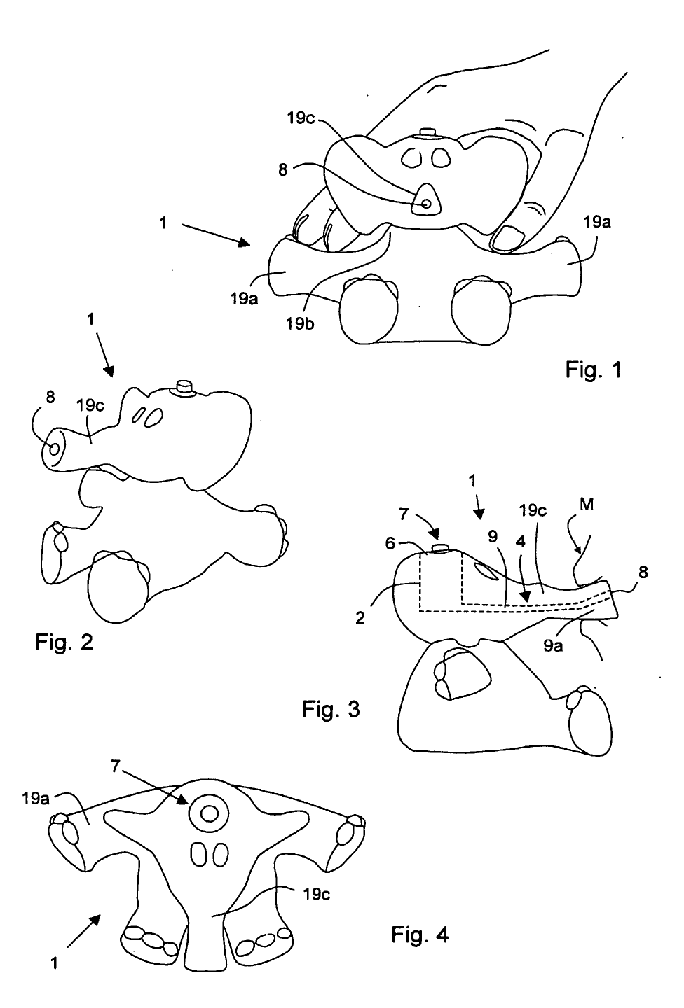

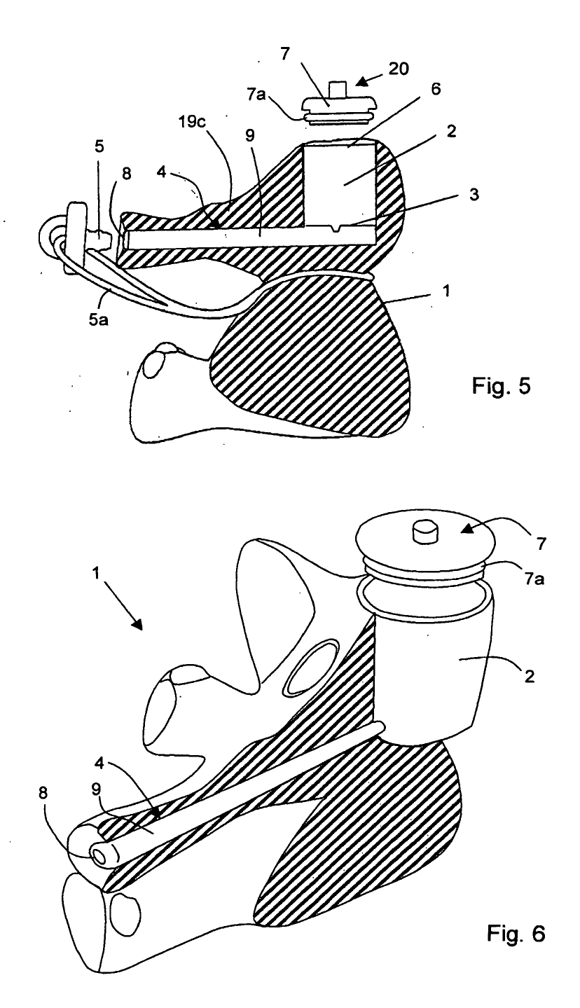

FIGS. 1-4 show different views of a device in accordance with the invention.

FIGS. 5, 6 show a side view and a perspective view of a device in accordance with the invention in partial section.

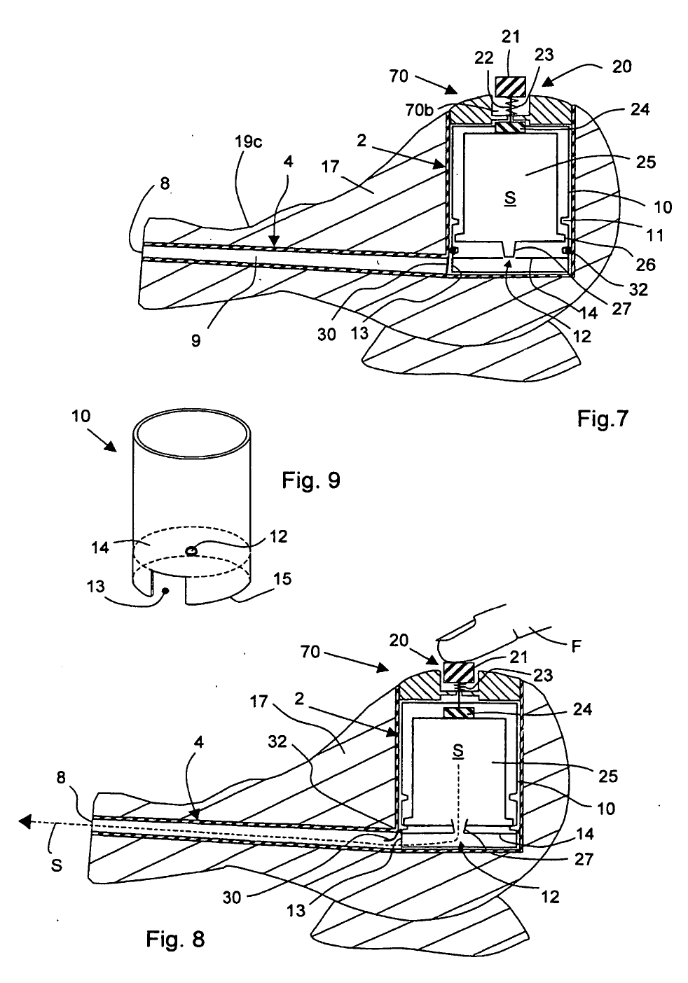

FIGS. 7, 8 show a sectional side view through a part of a device in accordance with the invention with a gas container inserted in an insert and with a transport device (non actuated: FIG. 7; actuated: FIG. 8).

FIG. 9 shows the insert according to FIGS. 7, 8.

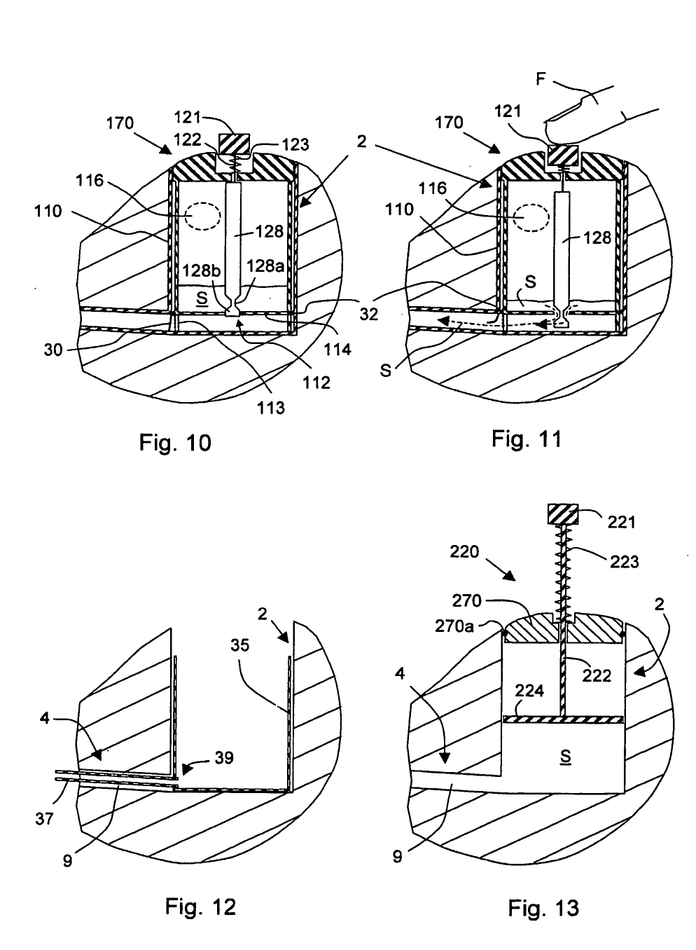

FIGS. 10, 11 show a section through a part of a device in accordance with the invention with a liquid substance inserted in an insert and with a transport device (non actuated: FIG. 10; actuated: FIG. 11).

FIG. 12 shows a section through a part of a device in accordance with the invention with an insert for the receiving unit and one for the dispensing unit.

FIG. 13 shows a section through a part of a device in accordance with the invention with a transport device surrounding a piston.

FIGS. 1 to 4 show a device 1 in accordance with the invention in different perspectives that has the outer shape of a sitting elephant. In FIG. 1 device 1 is shown grasped by a hand and the fingers of the hand grasp the elephant on its front feet 19a and in the neck and ear range 19b. Receiving unit 2 schematically shown in FIG. 3 and designed in a substantially cylindrical manner is arranged in the area of the elephant's head. A substantially horizontal hollow line 9 starts from the lower area of receiving unit 2, runs in trunk 19c of the elephant and is provided on its free end with dispensing opening 8. A receiving opening 6 in the upper area of receiving unit 2 can be reversibly closed with a first closure unit 7. When closure unit 7 is removed, receiving unit 2 can be filled with a liquid substance. Alternatively, a gas container can be inserted in receiving unit 2 for which an appropriately designed fitting device 3 is provided (indicated only schematically).

Transport device 20 is integrated into first closure unit 7 with which device a certain amount of substance can be administered from receiving unit 2 or a container inserted therein via hollow line 9 and dispensing opening 9. Hollow line section 9a arranged directly in front of dispensing opening 9 runs obliquely upwards when device 1 has been placed on a horizontal surface in order to prevent the substance from running out and/or dropping out of device 1.

FIG. 3 schematically indicates that according to an especially preferred embodiment of the invention an oral administration can take place. In the example shown a child takes trunk 19c into his mouth M while his schematically represented lips preferably surround trunk 19c including the dispensing opening. Device 1 should be tilted, if necessary, towards the face during the administration in order that in particular a liquid substance can completely pass into mouth M.

Device 1 shown in FIGS. 5, 6 is designed in a manner quite similar to that of FIGS. 1-4. First closure unit 7 comprises sealing ring 7a on the bottom on its circumference, which ring seals receiving opening 6 of receiving unit 2 and imparts a reliable seat to closure unit 7. A second closure unit 5 is designed as a pacifier and connected to the elephant's body by loss guard 5a designed as a band, chain or the like (here as a neck band).

The first and/or the second closure unit 7, 5 can also comprise an inner or outer threading that can accordingly be screwed to an outer threading or an inner threading in the area of receiving opening 6 or of dispensing unit 8. Other closure units are also possible, e.g., closure units that can be closed and opened by shifting, engaging or by some other actuation.

Receiving unit 2 and dispensing unit 4 are designed in one piece in accordance with the exemplary embodiment shown in FIGS. 5, 6 and advantageously manufactured from a food-resistant material, preferably an appropriate plastic, high-grade steel or aluminum. Alternatively, the inner surfaces of receiving unit 2 and of dispensing unit 4 can be provided with a coating of a food-resistant material.

Plastic material 17 is arranged positively and non-positively around receiving unit 2 and dispensing unit 4. Plastic 17 has the outer contour of device 1 of the invention, that is, in the present instance that of a toy elephant. Plastic 17 is preferably applied around receiving unit 2 and dispensing unit 4 by an injection-molding method, blow-out method or rotation method. A relatively soft plastic is preferably used in order that the toy-like nature of the device is more evident and that it is better accepted emotionally by children.

FIGS. 7 and 8 show a section of a device in accordance with the invention in section, again designed as an elephant figure, for administering a gaseous substance S. A substantially cylindrical insert 10 is inserted in receiving unit 2 into which insert a likewise substantially cylindrical gas container 25 is placed. Substance S to be administered, e.g., an aerosol, is enclosed in gas container 25. In addition, transport device 20 is integrated into first closure unit 70 in order to be able to administer substance S actively and, if necessary, in a precisely dosed amount. The first closure unit 70 is screwed in the instance shown to the upper edge of receiving unit 2 (indicated only schematically).

FIG. 9 shows insert 10 more precisely. It is open at the top in order to be able to insert container 25. In addition, insert 10 comprises intermediate bottom 14 just above its lower edge 15. This intermediate bottom 14 comprises central opening 12 that is advantageously lowered (intermediate bottom 14 with lowered opening 12 corresponds to fitting device 3 indicated only schematically in FIG. 3). Recess 13 is provided in the wall section between intermediate bottom 14 and lower edge 15. Moreover, two annular grooves 11 spaced at an interval in the longitudinal direction of insert 10 and inwardly directed are provided above intermediate bottom 14 (see FIGS. 7, 8; omitted in FIG. 9 for the sake of clarity).

Gas container 25, that is filled with the gas or aerosol, preferably commercially available and filled, e.g., with an asthma spray, is designed to be inserted into insert 10. Lower edge 26 of container 25 is widened out on the circumferential side in such a manner that it is slightly wider than the inner diameter of the two annular grooves 11 of insert 10. Thus, container 25 must be inserted (and removed) into and out of insert 10 with a slight application of force in order that widened-out edge 26 can overcome the narrow point defined by upper annular groove 11. Container 25 is subsequently supported in insert 10 where its freedom of play is given in the axial direction by the interval of the two annular grooves 11. Container 25 comprises central valve 27 on its bottom that opens under pressure and allows gaseous substance S to escape. Valve 27 is arranged centrally and above opening 12 of intermediate bottom 14 when container 25 is inserted.

Already-cited transport device 20 is provided in order to administer substance S. It comprises in the exemplary embodiment according to FIGS. 7, 8 pushbutton 21 connected to rod 22 whose other free end is fastened to stamp 24. Rod 22 extends through a central recess in first closure unit 70 and is surrounded in the upper section by spiral spring 23 that serves to exert pressure from below on pushbutton 21 and is supported in recess 70b of closure unit 70. Stamp 24 is in contact with the top of gas container 25.

When the pushbutton is actuated with a finger F (see FIG. 8), stamp 24 is pressed down against the spring force of spring 21 and therewith container 25 is pressed down also, which opens valve 27, as is indicated only schematically, and allows gas to pass from container 25 through opening 12 in intermediate bottom 14, through recess 13 (shown in dotted lines in FIGS. 7, 7) and through hollow line 9 to dispensing opening 8. Container 24 is brought back into its initial position (see FIG. 7) after pushbutton 21 is let loose by means of an internal, not-shown spring in container 25 or also by return means (also not shown) arranged in insert 10.

A thin membrane functioning as flow regulating means 30 is arranged in the transition area from receiving unit 2 to hollow line 9. This membrane blocks hollow line 9 when transport device 20 is not actuated (FIG. 7) whereas when transport device 20 is actuated, the membrane is raised by the gas flow (FIG. 8). The membrane can also be raised by suction from dispensing opening 8. Such a membrane 30 can be advantageous, if other blocking means are not present, in particular when administering liquids.

Sealing means 32 designed as an O-ring is provided between the outer wall of insert 10 and the inner wall of receiving unit 2 directly above hollow line 9 in lower annular groove 11. This sealing means prevents a penetration of the gas (FIGS. 7, 8) or of a liquid to be administered (FIGS. 10, 11) into the upper part of receiving unit 2.

FIGS. 10, 11 show an example of administering a liquid. To this end the device according to FIGS. 7, 8 is also preferably used, but advantageously with another first closure unit 170 and another insert 110, which closure unit 170 and insert 110 are designed in one piece in the present instance. Insert 110 is again designed substantially cylindrically and comprises, like the insert according to FIG. 9, an intermediate bottom 114, central opening 112 and a lower recess 113. Moreover, opening 116, indicated in dotted lines, is provided in an upper area of insert 110 in order to pour a liquid substance S into the inner chamber of the insert.

In the embodiment according to FIGS. 10, 11 a substantially elongated cylinder 128 is provided instead of a stamp. Cylinder 128 is connected at one of its ends to rod 122 fastened for its part (see FIGS. 7, 8) to pushbutton 121 loaded by spring 123. Constriction 128a on the circumferential side is provided in the area of the other end of cylinder 128. In contrast thereto, free end 128b of cylinder 128 widens out again and has a slightly larger diameter than opening 112 in intermediate bottom 114. Free end 128b can also be designed to be conical in order to assure a better seal of opening 112 when pushbutton 121 is not actuated, see FIG. 10. Thus, cylinder 128 constitutes a valve designed as a flow regulating means similar to valve 27 for gas is according to FIGS. 7, 8.

In order to use the device according to FIGS. 10, 11 a liquid substance S is filled through opening 116 into the inner chamber of insert 110 when insert 110 (including closure unit 170, pushbutton 121, cylinder 128) has been removed from receiving unit 2, during which pushbutton 121 is not actuated. Then, insert 110 is inserted into receiving unit 2 and closure unit 170 screwed on. Dispensing opening 8 can now be introduced into the mouth of the child and pushbutton 121 pressed down. Substance S now flows through the opening annular slot between constriction 128a and opening 112 and can be sucked up by the child. Alternatively or additionally, the entire device can be tilted in order that substance S can flow to dispensing opening 8. Even this embodiment thus makes use of a valve action so that membrane 30, that is also shown, is likewise not absolutely necessary. A difference between the two embodiments according to FIGS. 7 and 8 on the one hand and FIGS. 10 and 11 on the other hand consists in that in the case of the latter the liquid substance S is not actively transported when pushbutton 121 is actuated but rather only opening 112 is freed for the liquid.

In the embodiment according to FIG. 12 a cylindrical insert 35 is placed into receiving unit 2. In addition, a second insert 37 is pushed into hollow line 9 of dispensing opening 4 from dispensing opening 8. The second insert 37 comprises an outer thread on its end facing dispensing unit 4 which thread is screwed to a corresponding inner thread in lower area 39 of first insert 35. Other types of connection are also possible, e.g., a plug connection or a bayonet closure. A substance to be administered makes contact only with replaceable first and second inserts 35, 37 here. Any closure unit for receiving unit 2 and/or dispensing unit 4 as well as any transport devices are not shown.

FIG. 13 shows another embodiment designed in particular for the administration of pulpy foods S. The same device 1 as previously described can be used here. However, a transport device 220 with a piston 224 integrated into a first closure unit 270 is provided here which piston is fastened to piston rod 222 whose other free end extends beyond closure unit 270 and is fastened to pushbutton 221 loaded by spring 223. For administration, the pap is placed into receiving unit 2 and closure unit 270 placed on; rubber ring 270a is provided for security. A slow pressing down of pushbutton 221 causes piston 224 to move downward and presses pulpy substance S through hollow line 9 to the dispensing opening.

The surfaces of the elements shown in the figures that come in contact with a substance to be administered are preferably manufactured from food-resistant materials or have a food-resistant coating. Appropriate plastic, glass, porcelain, high-great steel and aluminum are particularly suitable to this end. The device is preferably designed to be washing-machine-resistant.

The invention is not limited to the exemplary embodiment shown in the figures. For example, more than one receiving unit and/or one dispensing unit can also be provided. Several hollow lines and several dispensing openings can also be present.

Thus, the device in accordance with the invention makes it possible to dispense liquid, gaseous or also powdery medicaments and foods including children's pap and other thick and foods in a manner which is simple and in particular suitable for children.

1. A device for administering or for ingesting fluid or powdery substances (S) to a person, especially medical substances and/or food to children, with a receiving unit (2) for receiving the substance (S) to be administered and with a dispensing unit (4) connected to the receiving unit (2) which dispensing unit comprises a dispensing opening (8) for dispensing the substance (S) to the person, which device (1) has at least in parts an outer form not directly functionally related to the administration.

2-44 (canceled)
